(19) United States
(12) Patent Application Publication
(10) Pub. No.: US 2025/0049005 A1
(43) Pub. Date: Feb. 13, 2025

(54) TRANSGENIC PRISTELLA

(71) Applicant: GLOFISH, LLC, Earth City, MO (US)

(72) Inventor: Aidas NASEVICIUS, Tampa, FL (US)

(21) Appl. No.: 18/716,574

(22) PCT Filed: Nov. 21, 2022
(86) PCT No.: PCT/US2022/080233

§ 371 Date: Jun. 5, 2024

Related U.S. Application Data
(60) Provisional application No. 63/287,153, filed on Dec. 8, 2021.

(51) Int. Cl. A01K 67/0278

(52) U.S. Cl. CPC A01K 67/0278; A01K 2217/052; A01K 2227/40

(57) ABSTRACT

The present invention relates to transgenic ornamental fish, as well as methods of making such fish by in vitro fertilization techniques. Also disclosed are methods of establishing a population of such transgenic fish and methods of providing them to the ornamental fish industry for the purpose of marketing.

### CROSS-REFERENCE TO RELATED APPLICATION

This application is being filed on Nov. 21, 2022, as a PCT International Patent Application and claims priority to and the benefit of U.S. Provisional Application No. 63/287,153, filed Dec. 8, 2021, which is incorporated by reference herein in its entirety.

### FIELD OF THE INVENTION

This invention relates to transgenic fish, particularly green, orange, purple and red transgenic Pristella.

### INTRODUCTION

Transgenic technology involves the transfer of a foreign gene into a host organism enabling the host to acquire a new and inheritable trait. Transgenic technology has many potential applications. For example, it can be used to introduce a transgene into a fish in order to create new varieties of fish. There are many ways of introducing a foreign gene into fish, including: microinjection (e.g., Zhu et al., 1985; Du et al., 1992), electroporation (Powers et al., 1992), sperm-mediated gene transfer (Khoo et al., 1992; Sin et al., 1993), gene bombardment or gene gun (Zelenin et al., 1991), liposome-mediated gene transfer (Szelei et al., 1994), and the direct injection of DNA into muscle tissue (Xu et al., 1999).

The first transgenic fish report was published by Zhu et al., (1985) using a chimeric gene construct consisting of a mouse metallothionein gene promoter and a human growth hormone gene. Most of the early transgenic fish studies have concentrated on growth hormone gene transfer with an aim of generating fast growing fish. While a majority of early attempts used heterologous growth hormone genes and promoters and failed to produce these fish (e.g. Chourrout et al., 1986; Penman et al., 1990; Brem et al., 1988; Gross et al., 1992), enhanced growth of transgenic fish has been demonstrated in several fish species including Atlantic salmon, several species of Pacific salmons, and loach (e.g. Du et al., 1992; Delvin et al., 1994, 1995; Tsai et al., 1995).

Pristella maxillaris, one of two species in the genus Pristella, is commonly known as the X-ray fish or X-ray tetra because of its translucent body. It is a widely distributed and adaptable fish, found in the Amazon and Orinoco basins, as well as coastal rivers in the Guianas in both acidic and alkaline waters. Unlike most other characins, it is tolerant of (and sometimes found in) slightly brackish water. It is small (up to around 5 cm or 2.0 in in length) and lives in large groups, and males can be distinguished from females by being smaller and thinner than the females. Like most other tetras, it feeds primarily on small insects and planktonic animals.

In the residential fish market, Pristella maxillaris is a small, adaptable fish. It is tolerant of a range of water chemistry values (i.e. pH 6-8). As a schooling species, it is usually kept in groups of at least six specimens and away from aggressive or predatory tank mates, but is otherwise easily kept in the community tank.

However, the availability of such Pristella having modified pigmentation for transgenesis with fluorescent proteins would result in better products for the ornamental fish industry due to better visualization of the various colors.

Many fluorescent proteins are known in the art and have been used to investigate various cellular processes, including fluorescent proteins exhibiting various green, red, pink, yellow, orange, blue, or purple colors. Although transgenic experiments involving fluorescent proteins have provided new markers and reporters for transgenesis, progress in the field of developing and producing Pristellas that express such proteins has been limited.

### Transgenic Pristella

In certain embodiments, the present disclosure concerns making transgenic fluorescent fish and providing such fish to the ornamental fish industry.

In some embodiments, transgenic fish or methods of making transgenic fish are provided. In certain aspects, the transgenic fish are fertile, transgenic, fluorescent fish. In a particular embodiment, the fish for use with the disclosed constructs and methods is the Pristella. Pristella skin color is determined by pigment cells in the skin, which contain pigment granules called melanosomes (black or brown color), xanthosomes (yellow color), erythrosomes (orange or red color), or iridosomes (iridescent colors, including white color). The number, size, and density of the pigment granules per pigment cell influence the color of the fish skin.

In certain specific embodiments, there are provided transgenic Pristella or progeny thereof comprising specific transgenic integration events, referred to herein as transformation events. These fish are of particular interest because, for example, they embody an aesthetically pleasing Green color. Transgenic fish comprising these specific transgenic events may be homozygous or heterozygous (including, for example, hemizygous) for the transformation event. Homozygous fish bred with fish lacking a transformation event will in nearly all cases produce 100% heterozygous offspring. Germ cells, eggs, sperm, and embryos comprising these specific transgenic events are also included as part of the invention.

In one such embodiment regarding a specific transgenic integration event, a green transgenic Pristella or progeny thereof is provided comprising chromosomally integrated transgenes, wherein the Pristella comprises the “Green Pristella 1 transformation event”, cryopreserved sperm comprising the Green Pristella 1 transformation event having been deposited at the American Type Culture Collections (ATCC), Historic District, 10801 University Blvd, Manassas, VA 20110 as Accession No. (PTA-127162). The chromosomally integrated transgenes may be present on one integrated expression cassette or two or more integrated expression cassettes. In certain aspects, such a transgenic Pristella is a fertile, transgenic Pristella. Such a transgenic Pristella may be homozygous or heterozygous (including, for example, hemizygous) for the transgenes or integrated expression cassette(s).

Also disclosed are methods of providing a transgenic Pristella comprising the Green Pristella 1 transformation event to the ornamental fish market. In some embodiments, the method comprises obtaining a transgenic Pristella or progeny thereof comprising chromosomally integrated transgenes, wherein the Pristella comprises the “Green Pristella 1 transformation event”, cryopreserved sperm comprising the Green Pristella 1 transformation event having been deposited at the ATCC, Historic District, 10801 University Blvd, Manassas, VA 20110 as Accession No. (PTA-127162), and distributing the fish to the ornamental fish market. Such fish may be distributed by a grower to a commercial distributor, or such fish may be distributed by a grower or a commercial distributor to a retailer such as, for example, a multi-product retailer having an ornamental fish department.

In some aspects, methods of producing a transgenic Pristella are provided comprising: (a) obtaining a Pristella that exhibits fluorescence and comprises one or more chromosomally integrated transgenes or expression cassettes, wherein the Pristella comprises the “Green Pristella 1 transformation event”, cryopreserved sperm comprising the Green Pristella 1 transformation event having been deposited at the ATCC, Historic District, 10801 University Blvd, Manassas, VA 20110 as Accession No. (PTA-127162); and (b) breeding the obtained Pristella with a second Pristella to provide a transgenic Pristella comprising the Green Pristella 1 transformation event. The second Pristella may be a transgenic or non-transgenic Pristella.

In further embodiments, also provided are methods of producing a transgenic organism, the method comprising using germ cell transplantation to produce the Green Pristella 1 transformation, such cryopreserved sperm having been deposited at the ATCC, Historic District, 10801 University Blvd, Manassas, VA 20110 as Accession No. (PTA-127162), to produce transgenic offspring. Such offspring may be, for example, a Pristella, a species of the Characidae family, a fish species or genus related to Pristella, or another fish species or genus.

In other embodiments, there are provided transgenic Pristella or progeny thereof comprising specific transgenic integration events, referred to herein as transformation events. These fish are of particular interest because, for example, they embody an aesthetically pleasing Orange color. Transgenic fish comprising these specific transgenic events may be homozygous or heterozygous (including, for example, hemizygous) for the transformation event. Homozygous fish bred with fish lacking a transformation event will in nearly all cases produce 100% heterozygous offspring. Germ cells, eggs, sperm, and embryos comprising these specific transgenic events are also included as part of the invention.

In one such embodiment regarding a specific transgenic integration event, an orange transgenic Pristella or progeny thereof is provided comprising chromosomally integrated transgenes, wherein the Pristella comprises the “Orange Pristella 1 transformation event”, cryopreserved sperm comprising the Orange Pristella 1 transformation event having been deposited at the ATCC, Historic District, 10801 University Blvd, Manassas, VA 20110 as Accession No. (PTA-127164). The chromosomally integrated transgenes may be present on one integrated expression cassette or two or more integrated expression cassettes. In certain aspects, such a transgenic Pristella is a fertile, transgenic Pristella. Such a transgenic Pristella may be homozygous or heterozygous (including, for example, hemizygous) for the transgenes or integrated expression cassette(s).

Also disclosed are methods of providing a transgenic Pristella comprising the Orange Pristella 1 transformation event to the ornamental fish market. In some embodiments, the method comprises obtaining a transgenic Pristella or progeny thereof comprising chromosomally integrated transgenes, wherein the Pristella comprises the “Orange Pristella 1 transformation event”, cryopreserved sperm comprising the Orange Pristella 1 transformation event having been deposited at the ATCC, Historic District, 10801 University Blvd, Manassas, VA 20110 as Accession No. (PTA-127164), and distributing the fish to the ornamental fish market. Such fish may be distributed by a grower to a commercial distributor, or such fish may be distributed by a grower or a commercial distributor to a retailer such as, for example, a multi-product retailer having an ornamental fish department.

In some aspects, methods of producing a transgenic Pristella are provided comprising: (a) obtaining a Pristella that exhibits fluorescence and comprises one or more chromosomally integrated transgenes or expression cassettes, wherein the Pristella comprises the “Orange Pristella 1 transformation event”, cryopreserved sperm comprising the Orange Pristella 1 transformation event having been deposited at the ATCC, Historic District, 10801 University Blvd, Manassas, VA 20110 as Accession No. (PTA-127164); and (b) breeding the obtained Pristella with a second Pristella to provide a transgenic Pristella comprising the Orange Pristella 1 transformation event. The second Pristella may be a transgenic or non-transgenic Pristella.

In further embodiments, also provided are methods of producing a transgenic organism, the method comprising using germ cell transplantation to produce the Orange Pristella 1 transformation, such cryopreserved sperm having been deposited at the (PTA-127164), to produce transgenic offspring. Such offspring may be, for example, a Pristella, a species of the Characidae family, a fish species or genus related to Pristella, or another fish species or genus.

In alternative embodiments, there are provided transgenic Pristella or progeny thereof comprising specific transgenic integration events, referred to herein as transformation events. These fish are of particular interest because, for example, they embody an aesthetically pleasing Purple color. Transgenic fish comprising these specific transgenic events may be homozygous or heterozygous (including, for example, hemizygous) for the transformation event. Homozygous fish bred with fish lacking a transformation event will in nearly all cases produce 100% heterozygous offspring. Germ cells, eggs, sperm, and embryos comprising these specific transgenic events are also included as part of the invention.

In one such embodiment regarding a specific transgenic integration event, a purple transgenic Pristella or progeny thereof is provided comprising chromosomally integrated transgenes, wherein the Pristella comprises the “Purple Pristella 1 transformation event”, cryopreserved sperm comprising the Purple Pristella 1 transformation event having been deposited at the ATCC, Historic District, 10801 University Blvd, Manassas, VA 20110 as Accession No. (PTA-127163). The chromosomally integrated transgenes may be present on one integrated expression cassette or two or more integrated expression cassettes. In certain aspects, such a transgenic Pristella is a fertile, transgenic Pristella. Such a transgenic Pristella may be homozygous or heterozygous (including, for example, hemizygous) for the transgenes or integrated expression cassette(s).

Also disclosed are methods of providing a transgenic Pristella comprising the Purple Pristella 1 transformation event to the ornamental fish market. In some embodiments, the method comprises obtaining a transgenic Pristella or progeny thereof comprising chromosomally integrated transgenes, wherein the Pristella comprises the “Purple Pristella 1 transformation event”, cryopreserved sperm comprising the Purple Pristella 1 transformation event having been deposited at the ATCC, Historic District, 10801 University Blvd, Manassas, VA 20110 as Accession No. (PTA-127163), and distributing the fish to the ornamental fish market. Such fish may be distributed by a grower to a commercial distributor, or such fish may be distributed by a grower or a commercial distributor to a retailer such as, for example, a multi-product retailer having an ornamental fish department.

In some aspects, methods of producing a transgenic Pristella are provided comprising: (a) obtaining a Pristella that exhibits fluorescence and comprises one or more chromosomally integrated transgenes or expression cassettes, wherein the Pristella comprises the “Purple Pristella 1 transformation event”, cryopreserved sperm comprising the Purple Pristella 1 transformation event having been deposited at the ATCC, Historic District, 10801 University Blvd, Manassas, VA 20110 as Accession No. (PTA-127163); and (b) breeding the obtained Pristella with a second Pristella to provide a transgenic Pristella comprising the Purple Pristella 1 transformation event. The second Pristella may be a transgenic or non-transgenic Pristella.

In further embodiments, also provided are methods of producing a transgenic organism, the method comprising using germ cell transplantation to produce the Purple Pristella 1 transformation, such cryopreserved sperm having been deposited at the ATCC, Historic District, 10801 University Blvd, Manassas, VA 20110 as Accession No. (PTA-127163), to produce transgenic offspring. Such offspring may be, for example, a Pristella, a species of the Characidae family, a fish species or genus related to Pristella, or another fish species or genus.

In other exemplary embodiments, there are provided transgenic Pristella or progeny thereof comprising specific transgenic integration events, referred to herein as transformation events. These fish are of particular interest because, for example, they embody an aesthetically pleasing Red color. Transgenic fish comprising these specific transgenic events may be homozygous or heterozygous (including, for example, hemizygous) for the transformation event. Homozygous fish bred with fish lacking a transformation event will in nearly all cases produce 100% heterozygous offspring. Germ cells, eggs, sperm, and embryos comprising these specific transgenic events are also included as part of the invention.

In one such embodiment regarding a specific transgenic integration event, a red transgenic Pristella or progeny thereof is provided comprising chromosomally integrated transgenes, wherein the Pristella comprises the “Red Pristella 1 transformation event”, cryopreserved sperm comprising the Red Pristella 1 transformation event having been deposited at the ATCC, Historic District, 10801 University Blvd, Manassas, VA 20110 as Accession No. (PTA-127165). The chromosomally integrated transgenes may be present on one integrated expression cassette or two or more integrated expression cassettes. In certain aspects, such a transgenic Pristella is a fertile, transgenic Pristella. Such a transgenic Pristella may be homozygous or heterozygous (including, for example, hemizygous) for the transgenes or integrated expression cassette(s).

Also disclosed are methods of providing a transgenic Pristella comprising the Red Pristella 1 transformation event to the ornamental fish market. In some embodiments, the method comprises obtaining a transgenic Pristella or progeny thereof comprising chromosomally integrated transgenes, wherein the Pristella comprises the “Red Pristella 1 transformation event”, cryopreserved sperm comprising the Red Pristella 1 transformation event having been deposited at the ATCC, Historic District, 10801 University Blvd, Manassas, VA 20110 as Accession No. (PTA-127165), and distributing the fish to the ornamental fish market. Such fish may be distributed by a grower to a commercial distributor, or such fish may be distributed by a grower or a commercial distributor to a retailer such as, for example, a multi-product retailer having an ornamental fish department.

In some aspects, methods of producing a transgenic Pristella are provided comprising: (a) obtaining a Pristella that exhibits fluorescence and comprises one or more chromosomally integrated transgenes or expression cassettes, wherein the Pristella comprises the “Red Pristella 1 transformation event”, cryopreserved sperm comprising the Red Pristella 1 transformation event having been deposited at the ATCC, Historic District, 10801 University Blvd, Manassas, VA 20110 as Accession No. (PTA-127165); and (b) breeding the obtained Pristella with a second Pristella to provide a transgenic Pristella comprising the Red Pristella 1 transformation event. The second Pristella may be a transgenic or non-transgenic Pristella.

In further embodiments, also provided are methods of producing a transgenic organism, the method comprising using germ cell transplantation to produce the Red Pristella 1 transformation, such cryopreserved sperm having been deposited at the (PTA-127165), to produce transgenic offspring. Such offspring may be, for example, a Pristella, a species of the Characidae family, a fish species or genus related to Pristella, or another fish species or genus.

As used in this specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Any embodiment of any of the present methods, kits, and compositions may consist of or consist essentially of—rather than comprise/include/contain/have—the described features and/or steps. Thus, in any of the claims, the term “consisting of” or “consisting essentially of” may be substituted for any of the open-ended linking verbs recited above, in order to change the scope of a given claim from what it would otherwise be using the open-ended linking verb.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION

### Transgenic Fish

In some aspects, the present disclosure regards transgenic fish. Methods of making transgenic fish are described in, for example, U.S. Pat. Nos. 7,135,613; 7,700,825; 7,834,239, each of which is incorporated by reference in its entirety. For example, a transgenic green Pristella may be generated using at least one expression cassette encoding Green fluorescent protein (GFP), such as zsGreen1, afraGFP, WasCFP, NowGFP, cerFP505, pporGFP, Kohinoor, efasGFP, eechGFP1, UnaG, bfloGFPa1, LanFP1 and LanFP2. Alternatively, a transgenic orange Pristella may be generated using at least one expression cassette encoding yellow fluorescent protein (YFP), such as ZsYellow1, phiYFP, zFP538, mPapaya, and mBanana. Alternatively, a transgenic purple Pristella may be generated using at least one expression cassette encoding Purple fluorescent protein (PFP), such as FP635, Katushka2S, mKate2, mCherry2, mCherry, mKate-S158C, eqFP650, mPlum, jRed, and mRFP1. Alternatively, a transgenic red Pristella may be generated using at least one expression cassette encoding Red fluorescent protein (RFP), such as TurboRFP, DsRed2, tdTomato, dTomato, eqFP578, DsRed-Express, DsRed-Express2, TagRFP, TagRFP-T, RRvT, cgfTagRFP, mRuby3, mNectarine, meffRFP, and amilFP593.

It is preferred that fish belonging to species and varieties of fish of commercial value, particularly commercial value within the ornamental fish industry, be used. Such fish include but are not limited to Pristellas, catfish, zebrafish and other danios, medaka, carp, tilapia, goldfish, tetras, barbs, sharks (family cyprinidae, such as rainbow shark), angelfish, loach, koi, glassfish, discus, eel, goby, gourami, guppy, Xiphophorus, hatchet fish, Molly fish, or pangasius. A particular fish for use in the context of the present disclosure is a Pristella. Pristellas are increasingly popular ornamental animals and would be of added commercial value in various colors. Pristella embryos are easily accessible and nearly transparent. Pristella skin color is determined by pigment cells in the skin, which contain pigment granules called melanosomes. The number, size, and density of the melanosomes per pigment cell influence the color of the fish skin.

In commercial aquaculture, Pristellas are spawned naturally. Briefly, one or two breeding pairs of tetra should be placed in a 2.5-5 gallon tank with an artificial spawning mat. The water level in the tank should be at least \tilde{ }2-3 inches and kept at 75-85° F. Low salinity (conductivity 100-200 uS/cm) and slight acidity (\tilde{ }pH 6.9) promote spawning. The fish may be exposed to a natural or artificial light cycle; the photoperiod may start at 8 am and end at 10 pm. The following day, remove the adults and incubate at 75-85 degrees Fahrenheit until the fry hatch and become free-swimming (i.e. about 4 to 6 days). At this time the fry can be safely handled and can be transferred into grow-out vats or outside earthen ponds. It takes about four to six months for Pristellas to mature. In a related embodiment, line propagation is maintained by cryopreserved sperm.

### Fertilization from Frozen Sperm

Fish sperm freezing methods are well-known in the art; see, e.g., Walker and Streisinger (1983) and Draper and Moens (2007), both of which are incorporated herein by reference in their entireties. To obtain the transgenic fish disclosed herein, frozen tetra sperm may be used to fertilize eggs.

In an example embodiment, young adult male green Pristella is selected. It should be held by its back ventral side up. The ventral side should be blotted with a dry paper towel. Ventral fins may be pushed away from the anal opening. When dry, the sides of the male should be squeezed gently and the released sperm should be collected and transferred into cold L-15 cell culture medium. Pipette in and out a couple times to rinse out the sperm. The process should be repeated with several males—when well-conditioned, approximately 10 males per 50 μl of L-15 should be used. The goal is to collect at least 5-7 μl of sperm per 50 μl of L-15. After collection, the sperm should be frozen as soon as possible. It should be re-mixed immediately before freezing. For freezing, 30 μl of cryoprotectant solution (10% fat-free dry milk, 15% methanol, and 15% 2-methoxyethanol in L-15) should be transferred to a cryovial. 15 μl of sperm suspension should be added and mixed by pipetting 2-3 times. The cryovial should be closed, transferred into 15 ml centrifuge tube, and vertically embedded in dry ice. After 20 minutes, transfer the cryovials into liquid nitrogen. When convenient (when all the cryovials have been transferred or there is a long gap between the transfers) transfer the frozen cryotubes into a Dewar with liquid nitrogen for long term storage.

In example method of recovering a green Pristella line, one or two breeding pairs of tetra should be placed in a 2.5-5 gallon tank with an artificial spawning mat. The water level in the tank should be at least \tilde{ }2-3 inches and kept at 75-85° F. Low salinity (conductivity 100-200 uS/cm) and slight acidity (\tilde{ }pH 6.9) promote spawning. The fish may be exposed to a natural or artificial light cycle; the photoperiod may start at 8 am and end at 10 pm. The following morning, ovulating females should be collected (they readily release eggs when gently squeezed). The bellies of the females should be blotted damp-dry with a paper towel. The eggs should not be exposed to water as this will prevent fertilization. The eggs should be squeezed out onto a slightly concave surface by applying light pressure to the sides of the abdomen with a thumb and index finger and sliding the fingers to the genital pore. Ready to spawn females will release the eggs extremely easily, and care should be taken not to squeeze the eggs out while blotting the fish. Good eggs are yellowish and translucent; eggs that have remained in the female too long appear white and opaque. The females will release the eggs only for an hour or so. Eggs from several females may be pooled; the eggs can be kept unfertilized for several minutes. The sperm should be thawed at 30-33° C. in a water bath for 30 seconds. 70 μl room temperature L-15 medium solution should be added to the vial and mixed. The sperm is then immediately added to the eggs and gently mixed. The sperm and eggs are activated by adding 750 μl of fish water and mixing. The mixture should be incubated for 5 minutes at room temperature. The eggs then are transferred to small tanks where they are further cultured using regular protocol to obtain green Pristella progeny.

In an example embodiment, young adult male orange Pristella is selected. It should be held by its back ventral side up. The ventral side should be blotted with a dry paper towel. Ventral fins may be pushed away from the anal opening. When dry, the sides of the male should be squeezed gently and the released sperm should be collected and transferred into cold L-15 cell culture medium. Pipette in and out a couple times to rinse out the sperm. The process should be repeated with several males-when well-conditioned, approximately 10 males per 50 μl of L-15 should be used. The goal is to collect at least 5-7 μl of sperm per 50 μl of L-15. After collection, the sperm should be frozen as soon as possible. It should be re-mixed immediately before freezing. For freezing, 30 μl of cryoprotectant solution (10% fat-free dry milk, 15% methanol, and 15% 2-methoxyethanol in L-15) should be transferred to a cryovial. 15 μl of sperm suspension should be added and mixed by pipetting 2-3 times. The cryovial should be closed, transferred into 15 ml centrifuge tube, and vertically embedded in dry ice. After 20 minutes, transfer the cryovials into liquid nitrogen. When convenient (when all the cryovials have been transferred or there is a long gap between the transfers) transfer the frozen cryotubes into a Dewar with liquid nitrogen for long term storage.

In an example method of recovering an orange Pristella line, one or two breeding pairs of tetra should be placed in a 2.5-5 gallon tank with an artificial spawning mat. The water level in the tank should be at least \tilde{ }2-3 inches and kept at 75-85° F. Low salinity (conductivity 100-200 uS/cm) and slight acidity (\tilde{ }pH 6.9) promote spawning. The fish may be exposed to a natural or artificial light cycle; the photoperiod may start at 8 am and end at 10 pm. The following morning, ovulating females should be collected (they readily release eggs when gently squeezed). The bellies of the females should be blotted damp-dry with a paper towel. The eggs should not be exposed to water as this will prevent fertilization. The eggs should be squeezed out onto a slightly concave surface by applying light pressure to the sides of the abdomen with a thumb and index finger and sliding the fingers to the genital pore. Ready to spawn females will release the eggs extremely easily, and care should be taken not to squeeze the eggs out while blotting the fish. Good eggs are yellowish and translucent; eggs that have remained in the female too long appear white and opaque. The females will release the eggs only for an hour or so. Eggs from several females may be pooled; the eggs can be kept unfertilized for several minutes. The sperm should be thawed at 30-33° C. in a water bath for 30 seconds. 70 μl room temperature L-15 medium solution should be added to the vial and mixed. The sperm is then immediately added to the eggs and gently mixed. The sperm and eggs are activated by adding 750 μl of fish water and mixing. The mixture should be incubated for 5 minutes at room temperature. The eggs then are transferred to small tanks where they are further cultured using regular protocol to obtain orange Pristella progeny.

In an example embodiment, young adult male purple Pristella is selected. It should be held by its back ventral side up. The ventral side should be blotted with a dry paper towel. Ventral fins may be pushed away from the anal opening. When dry, the sides of the male should be squeezed gently and the released sperm should be collected and transferred into cold L-15 cell culture medium. Pipette in and out a couple times to rinse out the sperm. The process should be repeated with several males-when well-conditioned, approximately 10 males per 50 μl of L-15 should be used. The goal is to collect at least 5-7 μl of sperm per 50 μl of L-15. After collection, the sperm should be frozen as soon as possible. It should be re-mixed immediately before freezing. For freezing, 30 μl of cryoprotectant solution (10% fat-free dry milk, 15% methanol, and 15% 2-methoxyethanol in L-15) should be transferred to a cryovial. 15 μl of sperm suspension should be added and mixed by pipetting 2-3 times. The cryovial should be closed, transferred into 15 ml centrifuge tube, and vertically embedded in dry ice. After 20 minutes, transfer the cryovials into liquid nitrogen. When convenient (when all the cryovials have been transferred or there is a long gap between the transfers) transfer the frozen cryotubes into a Dewar with liquid nitrogen for long term storage.

In example method of recovering a purple Pristella line, one or two breeding pairs of tetra should be placed in a 2.5-5 gallon tank with an artificial spawning mat. The water level in the tank should be at least \tilde{ }2-3 inches and kept at 75-85° F. Low salinity (conductivity 100-200 uS/cm) and slight acidity (\tilde{ }pH 6.9) promote spawning. The fish may be exposed to a natural or artificial light cycle; the photoperiod may start at 8 am and end at 10 pm. The following morning, ovulating females should be collected (they readily release eggs when gently squeezed). The bellies of the females should be blotted damp-dry with a paper towel. The eggs should not be exposed to water as this will prevent fertilization. The eggs should be squeezed out onto a slightly concave surface by applying light pressure to the sides of the abdomen with a thumb and index finger and sliding the fingers to the genital pore. Ready to spawn females will release the eggs extremely easily, and care should be taken not to squeeze the eggs out while blotting the fish. Good eggs are yellowish and translucent; eggs that have remained in the female too long appear white and opaque. The females will release the eggs only for an hour or so. Eggs from several females may be pooled; the eggs can be kept unfertilized for several minutes. The sperm should be thawed at 30-33° C. in a water bath for 30 seconds. 70 μl room temperature L-15 medium solution should be added to the vial and mixed. The sperm is then immediately added to the eggs and gently mixed. The sperm and eggs are activated by adding 750 μl of fish water and mixing. The mixture should be incubated for 5 minutes at room temperature. The eggs then are transferred to small tanks where they are further cultured using regular protocol to obtain purple Pristella progeny.

In an example embodiment, young adult male red Pristella is selected. It should be held by its back ventral side up. The ventral side should be blotted with a dry paper towel. Ventral fins may be pushed away from the anal opening. When dry, the sides of the male should be squeezed gently and the released sperm should be collected and transferred into cold L-15 cell culture medium. Pipette in and out a couple times to rinse out the sperm. The process should be repeated with several males-when well-conditioned, approximately 10 males per 50 μl of L-15 should be used. The goal is to collect at least 5-7 μl of sperm per 50 μl of L-15. After collection, the sperm should be frozen as soon as possible. It should be re-mixed immediately before freezing. For freezing, 30 μl of cryoprotectant solution (10% fat-free dry milk, 15% methanol, and 15% 2-methoxyethanol in L-15) should be transferred to a cryovial. 15 μl of sperm suspension should be added and mixed by pipetting 2-3 times. The cryovial should be closed, transferred into 15 ml centrifuge tube, and vertically embedded in dry ice. After 20 minutes, transfer the cryovials into liquid nitrogen. When convenient (when all the cryovials have been transferred or there is a long gap between the transfers) transfer the frozen cryotubes into a Dewar with liquid nitrogen for long term storage.

In example method of recovering a red Pristella line, one or two breeding pairs of tetra should be placed in a 2.5-5 gallon tank with an artificial spawning mat. The water level in the tank should be at least \tilde{ }2-3 inches and kept at 75-85° F. Low salinity (conductivity 100-200 uS/cm) and slight acidity (\tilde{ }pH 6.9) promote spawning. The fish may be exposed to a natural or artificial light cycle; the photoperiod may start at 8 am and end at 10 pm. The following morning, ovulating females should be collected (they readily release eggs when gently squeezed). The bellies of the females should be blotted damp-dry with a paper towel. The eggs should not be exposed to water as this will prevent fertilization. The eggs should be squeezed out onto a slightly concave surface by applying light pressure to the sides of the abdomen with a thumb and index finger and sliding the fingers to the genital pore. Ready to spawn females will release the eggs extremely easily, and care should be taken not to squeeze the eggs out while blotting the fish. Good eggs are yellowish and translucent; eggs that have remained in the female too long appear white and opaque. The females will release the eggs only for an hour or so. Eggs from several females may be pooled; the eggs can be kept unfertilized for several minutes. The sperm should be thawed at 30-33° C. in a water bath for 30 seconds. 70 μl room temperature L-15 medium solution should be added to the vial and mixed. The sperm is then immediately added to the eggs and gently mixed. The sperm and eggs are activated by adding 750 μl of fish water and mixing. The mixture should be incubated for 5 minutes at room temperature. The eggs then are transferred to small tanks where they are further cultured using regular protocol to obtain red Pristella progeny.

Parichy and Johnson, 2001, which is incorporated by reference in its entirety, provides additional examples regarding in vitro fertilization.

The present disclosure further encompasses progeny of a transgenic fish containing the Green Pristella 1 transformation event, as well as such transgenic fish derived from a transgenic fish egg, sperm cell, embryo, or other cell containing a genomically integrated transgenic construct. “Progeny,” as the term is used herein, can result from breeding two transgenic fish of the invention, or from breeding a first transgenic fish of the invention to a second fish that is not a transgenic fish of the invention. In the latter case, the second fish can, for example, be a wild-type fish, a specialized strain of fish, a mutant fish, or another transgenic fish. The second fish may be of the same species, or may be of a different species or genus. The hybrid progeny of these matings have the benefits of the transgene for fluorescence combined with the benefits derived from these other lineages.

The simplest way to identify fish containing the Green Pristella 1 transformation event is by visual inspection, as the fish in question would be green colored and immediately distinguishable from non-transgenic fish.

The present disclosure further encompasses progeny of a transgenic fish containing the Orange Pristella 1 transformation event, as well as such transgenic fish derived from a transgenic fish egg, sperm cell, embryo, or other cell containing a genomically integrated transgenic construct. “Progeny,” as the term is used herein, can result from breeding two transgenic fish of the invention, or from breeding a first transgenic fish of the invention to a second fish that is not a transgenic fish of the invention. In the latter case, the second fish can, for example, be a wild-type fish, a specialized strain of fish, a mutant fish, or another transgenic fish. The second fish may be of the same species, or may be of a different species or genus. The hybrid progeny of these matings have the benefits of the transgene for fluorescence combined with the benefits derived from these other lineages.

The simplest way to identify fish containing the Orange Pristella 1 transformation event is by visual inspection, as the fish in question would be orange colored and immediately distinguishable from non-transgenic fish.

The present disclosure further encompasses progeny of a transgenic fish containing the Purple Pristella 1 transformation event, as well as such transgenic fish derived from a transgenic fish egg, sperm cell, embryo, or other cell containing a genomically integrated transgenic construct. “Progeny,” as the term is used herein, can result from breeding two transgenic fish of the invention, or from breeding a first transgenic fish of the invention to a second fish that is not a transgenic fish of the invention. In the latter case, the second fish can, for example, be a wild-type fish, a specialized strain of fish, a mutant fish, or another transgenic fish. The second fish may be of the same species, or may be of a different species or genus. The hybrid progeny of these matings have the benefits of the transgene for fluorescence combined with the benefits derived from these other lineages.

The simplest way to identify fish containing the Purple Pristella 1 transformation event is by visual inspection, as the fish in question would be purple colored and immediately distinguishable from non-transgenic fish.

The present disclosure further encompasses progeny of a transgenic fish containing the Red Pristella 1 transformation event, as well as such transgenic fish derived from a transgenic fish egg, sperm cell, embryo, or other cell containing a genomically integrated transgenic construct. “Progeny,” as the term is used herein, can result from breeding two transgenic fish of the invention, or from breeding a first transgenic fish of the invention to a second fish that is not a transgenic fish of the invention. In the latter case, the second fish can, for example, be a wild-type fish, a specialized strain of fish, a mutant fish, or another transgenic fish. The second fish may be of the same species, or may be of a different species or genus. The hybrid progeny of these matings have the benefits of the transgene for fluorescence combined with the benefits derived from these other lineages.

The simplest way to identify fish containing the Red Pristella 1 transformation event is by visual inspection, as the fish in question would be red colored and immediately distinguishable from non-transgenic fish. It should be appreciated that depending on the specific RFP used and/or the insertion location of the expression cassette, the transgenic red Pristella may have a color that is maintained over the course of the life of the transgenic red Pristella's life and/or throughout multiple generations. It should also be appreciated that depending on the specific RFP used and/or the insertion location of the expression cassette, the transgenic red Pristella may have a color that fades over the course of the transgenic red Pristella's life. For example, the red transgenic Pristella may change in color from red to pale red, or from red to pink. In addition, depending on the specific RFP used and/or the insertion location of the expression cassette, the transgenic red Pristella may have a color that fades over generations. For example, the red transgenic Pristella may change in color from one generation to the next, such that an older generation may exhibit the red color, but a younger generation may appear pale red, or pink.

### EXAMPLES

Certain embodiments of the invention are further described with reference to the following examples. These examples are intended to be merely illustrative of the invention and are not intended to limit or restrict the scope of the present invention in any way and should not be construed as providing conditions, parameters, reagents, or starting materials that must be utilized exclusively in order to practice the art of the present invention.

### Example 1—Green Transgenic Pristella

Transgenic fish exhibiting a Green color are provided. The specific transgenic events embodied in these fish are designated the “Green Pristella 1 transformation event”. Sperm from these fish may be used to fertilize tetra eggs and thereby breed transgenic tetra that comprise these specific transgenic integration events. Sperm from this line was deposited at the ATCC, Historic District, 10801 University Blvd, Manassas, VA 20110, under the provisions of the Budapest Treaty as “Green Pristella 1” (the deposit was designated as Accession No. (PTA-127162)).

The fluorescent transgenic fish have use as ornamental fish in the market. Stably expressing transgenic lines can be developed by breeding a transgenic individual with a wild-type fish, mutant fish, or another transgenic fish. The desired transgenic fish can be distinguished from non-transgenic fish by observing the fish in white light, sunlight, ultraviolet light, blue light, or any other useful lighting condition that allows visualization of the Green color of the transgenic fish.

### Example 2—Orange Transgenic Pristella

Transgenic fish exhibiting an Orange color are provided. The specific transgenic events embodied in these fish are designated the “Orange Pristella 1 transformation event”. Sperm from these fish may be used to fertilize tetra eggs and thereby breed transgenic tetra that comprise these specific transgenic integration events. Sperm from this line was deposited at the ATCC, Historic District, 10801 University Blvd, Manassas, VA 20110 under the provisions of the Budapest Treaty as “Orange Pristella 1” (the deposit was designated as Accession No. (PTA-127164)).

The fluorescent transgenic fish have use as ornamental fish in the market. Stably expressing transgenic lines can be developed by breeding a transgenic individual with a wild-type fish, mutant fish, or another transgenic fish. The desired transgenic fish can be distinguished from non-transgenic fish by observing the fish in white light, sunlight, ultraviolet light, blue light, or any other useful lighting condition that allows visualization of the Orange color of the transgenic fish.

### Example 3—Purple Transgenic Pristella

Transgenic fish exhibiting a Purple color are provided. The specific transgenic events embodied in these fish are designated the “Purple Pristella 1 transformation event”. Sperm from these fish may be used to fertilize tetra eggs and thereby breed transgenic tetra that comprise these specific transgenic integration events. Sperm from this line was deposited at the ATCC, Historic District, 10801 University Blvd, Manassas, VA 20110 under the provisions of the Budapest Treaty as “Purple Pristella 1” (the deposit was designated as Accession No. (PTA-127163)).

The fluorescent transgenic fish have use as ornamental fish in the market. Stably expressing transgenic lines can be developed by breeding a transgenic individual with a wild-type fish, mutant fish, or another transgenic fish. The desired transgenic fish can be distinguished from non-transgenic fish by observing the fish in white light, sunlight, ultraviolet light, blue light, or any other useful lighting condition that allows visualization of the Purple color of the transgenic fish.

### Example 4—Red Transgenic Pristella

Transgenic fish exhibiting a Red color are provided. The specific transgenic events embodied in these fish are designated the “Red Pristella 1 transformation event”. Sperm from these fish may be used to fertilize tetra eggs and thereby breed transgenic tetra that comprise these specific transgenic integration events. Sperm from this line was deposited at the ATCC, Historic District, 10801 University Blvd, Manassas, VA 20110 under the provisions of the Budapest Treaty as “Red Pristella 1” (the deposit was designated as Accession No. (PTA-127165)).

The fluorescent transgenic fish have use as ornamental fish in the market. Stably expressing transgenic lines can be developed by breeding a transgenic individual with a wild-type fish, mutant fish, or another transgenic fish. The desired transgenic fish can be distinguished from non-transgenic fish by observing the fish in white light, sunlight, ultraviolet light, blue light, or any other useful lighting condition that allows visualization of the Red color of the transgenic fish.

The fluorescent transgenic fish should also be valuable in the market for scientific research tools because they can be used for embryonic studies such as tracing cell lineage and cell migration. Additionally, these fish can be used to mark cells in genetic mosaic experiments and in fish cancer models.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

The above specification, examples and data provide a complete description of the manufacture and use of the composition of the invention. Since many embodiments of the invention can be made without departing from the spirit and scope of the invention, the invention resides in the claims hereinafter appended.

### REFERENCES

The following references, to the extent that they provide exemplary procedural or other details supplementary to those set forth herein, are specifically incorporated herein by reference.
U.S. Pat. No. 7,135,613
U.S. Pat. No. 7,700,825
U.S. Pat. No. 7,834,239
Brem et al., Aquaculture, 68:209-219, 1988.
Chourrout et al., Aquaculture, 51:143-150, 1986.
Delvin et al., Nature, 371:209-210, 1994.
Du et al., Bio Technology, 10:176-181, 1992.
Innes, W.T., Exotic Aquarium Fishes: A work of general reference, Innes Publishing
Company, Philadelphia, 1950.
Gross et al., Aquaculature, 103:253-273, 1992.
Khoo et al., Aquaculture, 107:1-19, 1992.
Lamason et al., Science, 310(5755):1782-1786, 2005.
Lee et al., PNAS, 110 (5) : 1640-1645, 2013.
Penman et al., Aquaculture, 85:35-50, 1990.
Powers et al., Mol. Marine Biol. Biotechnol., 1:301-308, 1992.
Seki et al., Scientific Reports, doi:10.1038/srep43185, 2017
Shagin et al., Mol Biol Evol. (5):841-50, 2004.
Sin et al., Aquaculture, 117:57-69, 1993.
Szelei et al., Transgenic Res., 3:116-119, 1994.
Tsai et al., Can. J. Fish Aquat. Sci., 52:776-787, 1995.
Xu et al., DNA Cell Biol., 18, 85-95, 1999.
Zelenin et al., FEBS Lett., 287(1-2):118-120, 1991.
Zhu et al., Z. Angew. Ichthyol., 1:31-34, 1985.

1. A transgenic Pristella comprising a chromosomally integrated expression cassette encoding a fluorescent protein, wherein the Pristella comprises the “Green Pristella 1 transformation event”.

2. The transgenic Pristella of claim 1, further defined as a fertile, transgenic Pristella.

3. The transgenic Pristella of claim 1, wherein the fish is homozygous for the integrated expression cassette.

4. The transgenic Pristella of claim 1, wherein the fish is heterozygous for the integrated expression cassette.

5. A method of providing a transgenic Pristella to the ornamental fish market, comprising obtaining a transgenic Pristella in accordance with claim 1, and distributing the fish to the ornamental fish market.

6. The method of claim 5, wherein the fish are distributed by a grower to a commercial distributor.

7. The method of claim 5, wherein the fish are distributed by a grower or a commercial distributor to a retailer.

8. The method of claim 7, wherein the retailer is a multi-product retailer having an ornamental fish department.

9. A method of producing a transgenic Pristella comprising:
(a) obtaining a Pristella that comprises a chromosomally integrated expression cassette encoding a fluorescent protein, wherein the Pristella comprises the “Green Pristella 1 transformation event”; and
(b) breeding the obtained Pristella with a second Pristella to provide a transgenic Pristella comprising the Green Pristella 1 transformation event.

10. The method of claim 9, wherein the second Pristella is a non-transgenic Pristella.

11. A progeny of a transgenic Pristella of claim 1 that comprises a chromosomally integrated expression cassette encoding a fluorescent protein, wherein the Pristella and progeny exhibit fluorescence and comprise the “Green Pristella 1 transformation event”.

12. The progeny fish of claim 11, further defined as a fertile, transgenic Pristella.

13. The progeny fish of claim 11, wherein the fish is homozygous for the integrated expression cassette.

14. The progeny fish of claim 11, wherein the fish is heterozygous for the integrated expression cassette.

15. A method of providing a transgenic fish to the ornamental fish market, comprising obtaining a progeny fish in accordance with claim 11, and distributing the fish to the ornamental fish market.

16. The method of claim 15, wherein the fish are distributed by a grower to a commercial distributor.

17. The method of claim 16, wherein the fish are distributed by a grower or a commercial distributor to a retailer.

18. The method of claim 17, wherein the retailer is a multi-product retailer having an ornamental fish department.

19. A method of producing a transgenic fish comprising:
(a) obtaining a transgenic fish in accordance with claim 1; and
(b) breeding the obtained fish with a second fish to provide a transgenic fish comprising the Green Pristella 1 transformation event.

20. The method of claim 19, wherein the second fish is a non-transgenic fish.

21.-80. (canceled)

81. The transgenic Pristella of claim 1, wherein cryopreserved sperm comprising the Green Pristella 1 transformation event having been deposited at the ATCC as Accession No. (PTA-127162).

82.-84. (canceled)

85. The transgenic Pristella of claim 9, wherein cryopreserved sperm comprising the Green Pristella 1 transformation event having been deposited at the ATCC as Accession No. (PTA-127162).

86. The transgenic Pristella of claim 11, wherein cryopreserved sperm comprising the Green Pristella 1 transformation event having been deposited at the ATCC as Accession No. (PTA-127162).
